(19) United States
(12) Patent Application Publication
(10) Pub. No.: US 2020/0297584 A1
(43) Pub. Date: Sep. 24, 2020

(54) System and Method for External Steering of Nasogastric Tube During Its Placement

(71) Applicant: Avent, Inc., Alpharetta, GA (US)

(72) Inventor: Daniel J. Rogers, Roswell, GA (US)

(21) Appl. No.: 16/360,124

(22) Filed: Mar. 21, 2019

(51) Int. Cl. A61J 15/00

(52) U.S. Cl. CPC A61J 15/0003; A61M 25/01; A61J 15/0011; A61J 15/0073

(57) ABSTRACT

A tubing assembly for use in conjunction with electronic catheter guidance systems is provided and includes a catheter and steering apparatus. The catheter has a proximal end and a distal end that define a lumen therebetween and extends in a longitudinal direction. The steering apparatus includes an electrical connection and a sheath. Further, the sheath includes an electroactive polymer layer, and the sheath's proximal end is coupled to the distal end of the electrical connection, where the steering apparatus is located within the lumen of the catheter. Activation of the electroactive polymer layer results in a change in dimension of the sheath, which initiates a change in a direction in which the catheter travels within a patient's body to assist in accurate placement of the catheter at a desired location. A catheter guidance system and a method for steering a catheter during its placement inside a body are also provided.

### BACKGROUND OF THE INVENTION

Physicians and other health care providers frequently use catheters to treat patients. Known catheters include a tube which is inserted into the human body. Certain catheters are inserted through the patient's nose or mouth for treating the gastrointestinal tract. These catheters, sometimes referred to as enteral catheters, typically include feeding tubes. The feeding tube lies in the stomach or intestines, and a feeding bag delivers liquid nutrient, liquid medicine or a combination of the two to the patient.

Other types of catheters are inserted into the patient's veins or arteries for treating the cardiovascular system. These catheters include, among others, the central venous catheter, peripheral venous catheter and the peripherally inserted central catheter (PICC). These catheters include a relatively small tube that passes through the patient's veins or arteries. The health care provider uses these catheters to provide patients with injections of medications, drugs, fluids, nutrients, or blood products over a period of time, typically several weeks or more.

When using these known catheters, it is important to place the end of the catheter at the proper location within the human body. Erroneous placement of the catheter tip may injure or harm the patient. For example, if the health care provider erroneously places an enteral catheter into the patient's lungs, liquid may be introduced into the lungs with harmful results. If the health care provider erroneously places a catheter into the wrong cavity of the cardiovascular system, the patient may experience infection or a harmful blockage.

While advancements have been made in the development of a signal generator placement control device for use in conjunction with electronic catheter guidance systems, there is still a risk of erroneous placement of a catheter by a health care provider, even when using a catheter guidance system. For instance, when a nasogastric (NG) tube is placed through the nasal cavity, the intent is for the NG tube to traverse through the esophagus, then down into the stomach, and into the small bowel, if desired. As the tube travels down the throat, the anatomy splits into the trachea and esophagus at the oropharynx. NG tubes can be misplaced into the trachea at this split, which can result in pneumonia, a pneumothorax, or even death. In addition, it is often challenging for health care providers to position the NG tube within the desired location in the gastrointestinal system, as the flexible nature of the NG tube makes manipulation of the tube into precise locations difficult.

Accordingly, there is a need for a system and method to overcome each of these disadvantages.

### SUMMARY OF THE INVENTION

Objects and advantages of the invention will be set forth in part in the following description, or may be obvious from the description, or may be learned through practice of the invention.

In one particular embodiment, the present invention is directed to a tubing assembly. The tubing assembly includes a catheter and a steering apparatus. The catheter has a proximal end and a distal end and extends in a longitudinal direction, where the proximal end and the distal end define a lumen therebetween. The steering apparatus includes an electrical connection having a distal end and a proximal end as well as a sheath having a proximal end and a distal end, where the sheath includes an electroactive polymer layer. The proximal end of the sheath is coupled to the distal end of the electrical connection, and the steering apparatus is located within the lumen of the catheter. Further, activation of the electroactive polymer layer results in a change in dimension of the sheath, where the change in dimension initiates a change in a direction in which the catheter travels within a body.

In one embodiment, the electroactive polymer layer can be an ionic electroactive polymer or an electric electroactive polymer.

In another embodiment, the sheath can include at least one insulating layer. For example, the sheath can include a first insulating layer and a second insulting layer, where the electroactive polymer layer can be disposed therebetween. Further, the insulating layer can include polyamide, polyethylene, polypropylene, poly-L-lysine, poly-D-lysine, polyethylene glycol, polyvinyl alcohol, polyvinyl acetate, polymethyl methacrylate, or a combination thereof.

In still another embodiment, the electrical connection can be configured to deliver an electrical signal from a power source to the electroactive polymer layer to initiate the change in dimension.

In yet another embodiment, the change in dimension of the sheath can result in the catheter changing in direction by an angle ranging from about 1° to about 180° relative to the longitudinal direction in which the catheter extends.

In one more embodiment, the proximal end of the electrical connection can be coupled to a controller coupler, where the controller coupler can be configured for connection to a power source.

In an additional embodiment, the electrical connection can include a wire.

In another embodiment, the tubing assembly can further include a signal generating assembly. The signal generating assembly can include an elongated wire assembly having a proximal end and a distal end and a signal generator, where the signal generator can be coupled to the distal end of the elongated wire assembly. Further, the elongated wire assembly can extend through a lumen of the sheath, and the signal generator can be positioned adjacent the distal end of the sheath towards the distal end of the catheter. In addition, the signal generator can be a magnetic field generator.

In another particular embodiment, the present invention is directed to a catheter guidance system. The system includes a controller, a power source, a tubing assembly, and a non-invasive movable receiver-transmitter or transceiver in communication with the tubing assembly, where the tubing assembly and the non-invasive movable receiver-transmitter or transceiver are electronically coupled to the controller. The tubing assembly includes a catheter and a signal generating assembly. The catheter includes a proximal end and a distal end and extends in a longitudinal direction, where the proximal end and the distal end define a lumen therebetween; and a steering apparatus, where the steering apparatus includes an electrical connection having a distal end and a proximal end and a sheath having a proximal end and a distal end and comprising an electroactive polymer layer, where the proximal end of the sheath is coupled to the distal end of the electrical connection, where the steering apparatus is located within the lumen of the catheter, where activation of the electroactive polymer layer from the power source via the electrical connection results in a change in dimension of the sheath, where the change in dimension initiates a change in direction in which the catheter travels within a body.

In one more embodiment of the present invention, a method for steering a catheter during placement of the catheter inside a body of a patient is provided. The method includes inserting a distal end of a tubing assembly into an orifice of the body, where the tubing assembly also has a proximal end and includes the catheter, where the catheter has a proximal end and a distal end and extends in a longitudinal direction, where the proximal end and the distal end define a lumen therebetween; and a steering apparatus, wherein the steering apparatus comprises an electrical connection having a distal end and a proximal end and a sheath having a proximal end and a distal end and comprising an electroactive polymer layer, where the proximal end of the sheath is coupled to the distal end of the electrical connection, where the steering apparatus is located within the lumen of the catheter. The method also includes connecting the tubing assembly to a power source; and delivering an electrical signal from the power source to the steering apparatus via the electrical connection to activate the electroactive polymer layer, where activating the electroactive polymer layer changes a dimension of the sheath, where the change in dimension changes a direction in which the catheter travels within the body.

In one embodiment, the orifice can be a nose or mouth.

In another embodiment, the change in dimension of the sheath can result in the catheter changing in direction by an angle ranging from about 1° to about 180° relative to the longitudinal direction in which the catheter extends.

In still another embodiment, the electroactive polymer layer can include an ionic electroactive polymer or an electric electroactive polymer.

In yet another embodiment, the method can include activating the electroactive polymer layer as the catheter reaches an area near the patient's epiglottis so that the catheter is steered towards the esophagus rather than the trachea.

In one more embodiment, the method can include activating the electroactive polymer layer as the catheter reaches an area near the patient's pylorus to facilitate duodenal or jejunal placement of the catheter.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a perspective view of the catheter position guidance system illustrating the display device, electronic catheter unit and hand-held transceiver being used to position a catheter within a patient in one embodiment of the present invention.

FIG. 2 is schematic block diagram of the electronic configuration of the catheter position guidance system illustrating the processor, memory device, signal generator, input devices and output devices in one embodiment of the present invention.

FIG. 3 is a top or plan view of the electronic catheter unit and the display device illustrating an enteral application involving a catheter inserted into a human body and indication of catheter information on the display device.

FIG. 4 is a top or plan view of the electronic catheter unit and the display device illustrating a parenteral application involving a catheter inserted into a human body and indication of catheter information on the display device.

FIG. 5 is a perspective view of the electronic catheter unit illustrating the tubing assembly and the signal generator being received by and housed in the tubing assembly in one embodiment of the present invention.

FIG. 6 is a perspective view of the distal end of the electronic catheter unit illustrating the steering apparatus in one embodiment of the present invention.

FIG. 7 is a cross-sectional view of the distal end of the electronic catheter unit of illustrating the steering apparatus in one embodiment of the present invention.

FIG. 8 is a perspective view of the signal generator illustrating the tubular insulator housing a portion of the wire assembly in one embodiment of the present invention.

FIG. 9 is a top or plan view of the of the electronic catheter unit illustrating an enteral application involving insertion of a catheter into a human body and the steering of the catheter down the esophagus upon activation of the steering apparatus in one embodiment of the present invention.

FIG. 10 is a top or plan view of the of the electronic catheter unit illustrating an enteral application involving insertion of a catheter into a human body and the steering of the catheter from the esophagus and into the stomach upon activation of the steering apparatus in one embodiment of the present invention.

FIG. 11 is a top or plan view of the of the electronic catheter unit illustrating an enteral application involving insertion of a catheter into a human body and the steering of the catheter from the stomach towards the pylorus upon activation of the steering apparatus in one embodiment of the present invention.

FIG. 12 is a top or plan view of the of the electronic catheter unit illustrating an enteral application involving insertion of a catheter into a human body and the steering of the catheter from the pylorus to the duodenum upon activation of the steering apparatus in one embodiment of the present invention.

FIG. 13 is a top or plan view of the of the electronic catheter unit illustrating an enteral application involving insertion of a catheter into a human body and the steering of the catheter from the duodenum to the jejunum upon activation of the steering apparatus in one embodiment of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

Generally speaking, the present invention is directed to a tubing assembly for use in conjunction with electronic catheter guidance systems. The tubing assembly includes a catheter and a steering apparatus. The catheter has a proximal end and a distal end that define a lumen therebetween and extends in a longitudinal direction. The steering apparatus includes an electrical connection and a sheath. Further, the sheath includes an electroactive polymer layer, and the sheath's proximal end is coupled to the distal end of the electrical connection, where the steering apparatus is located within the lumen of the catheter. Activation of the electroactive polymer layer results in a change in dimension of the sheath, which initiates a change in a direction in which the catheter travels within a patient's body to assist in accurate placement of the catheter at a desired location. A catheter guidance system and a method for steering a catheter during its placement inside a body are also provided. Because of the specific components of the tubing assembly, catheter guidance system, and their methods of use, the present inventor has found that the placement of a catheter with a patient's gastrointestinal tract or any other anatomical location can be precisely controlled when the electroactive polymer layer is activated.

For instance, a health care provider can use the tubing assembly and catheter guidance system of the present invention to manipulate the placement of a catheter from outside the body. Such manipulation is facilitated by the electroactive polymer layer that forms at least a part of the sheath of the steering apparatus of the tubing assembly, where the polymer or polymers in the electroactive polymer layer can exhibit a change in dimension (e.g., a change in size, shape, and/or diameter) when exposed to an electric field, where the electric field can be applied from a power source in the form of a voltage or current via an electrical connection between the power source and the electroactive polymer layer. Because of the change in dimension of the steering apparatus due to the changes to the electroactive polymer layer, the catheter, which surrounds the steering apparatus (i.e., the steering apparatus is disposed within the lumen of the catheter), can bend in a desired direction such that the catheter can be accurately placed in a specific anatomical region.

For example, a catheter extending in a longitudinal direction L can change its direction of travel by an angle θ ranging from about 1° to about 180°, such as from about 5° to about 160°, such as from about 10° to about 90°, or any ranges therebetween, relative to the longitudinal direction L in which the catheter extends in response to the activation and resulting change in dimension of the electroactive polymer layer in the sheath of the steering apparatus, where the steering apparatus can be positioned or disposed within the catheter's lumen. Further, the length L₁ of the sheath component of the steering apparatus relative to the overall length of the catheter can be relatively small, such as from about 0.5 inches (about 12.5 millimeters) to about 10 inches (about 250 millimeters), such as from about 1 inch (about 25 millimeters) to about 8 inches (about 200 millimeters), such as from about 2 inches (about 50 millimeters) to about 6 inches (about 150 millimeters), or any ranges therebetween. Nevertheless, despite the relatively short length of the sheath with respect to the overall catheter length, activation of the electroactive polymer layer is sufficient to adjust or alter the direction in which the catheter was traveling prior to activation.

The various components of the tubing assembly and catheter guidance system of the present invention are discussed in more detail below.

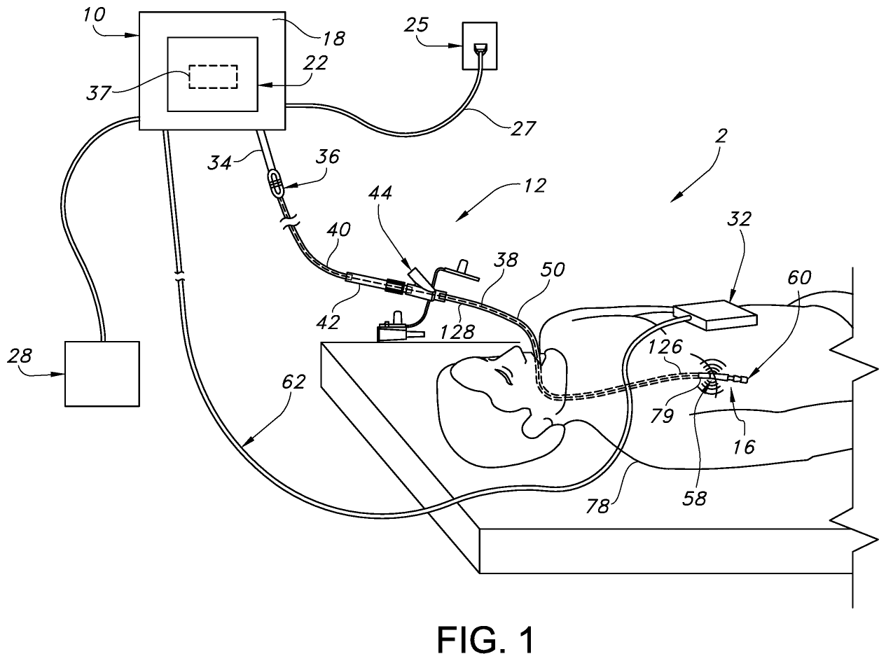

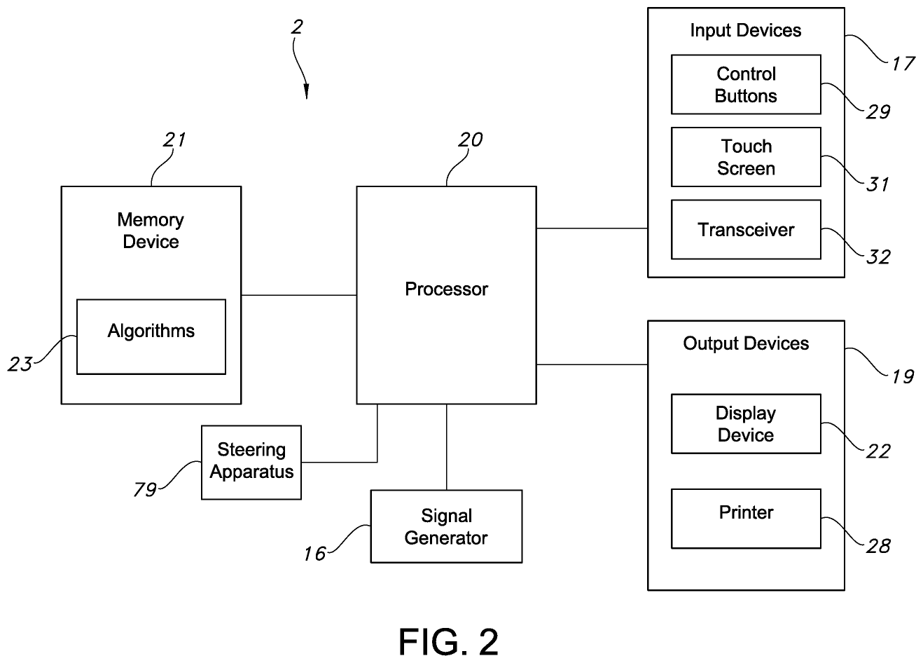

Referring now to the drawings, in an embodiment illustrated in FIGS. 1 and 2, the catheter position guidance system or catheter guidance system 2 can include: (a) an apparatus 10 having a housing 18 which supports a controller or processor 20 and a display device 22; (b) a non-invasive movable receiver-transmitter or transceiver 32 electronically coupled to the processor 20 by a wire, cable, signal data connection or signal carrier 62; (c) a power cord 27 that couples the apparatus 10 to a power source 25 (e.g., a power supply or battery); (d) a printer 28 coupled to the apparatus 10 for printing out paper having graphics which indicate catheter location information; and (e) an invasive electronic catheter unit 12 including a catheter 50, signal generator 58, and steering apparatus 79 in communication with the transceiver 32 and operatively coupled to the apparatus 10 by a wire, cable, cord or electrical extension 34, which, in turn, is operatively coupled to the processor 20, where it is also to be understood that the connection can be wireless (not shown). It should also be appreciated that the transceiver 32 can include a device which has a separate signal receiver and signal transmitter. The transceiver 32 can also include a single device which functions so as to receive and transmit signals.

As best illustrated in FIG. 2, the catheter position guidance system 2, in one embodiment, includes: (a) a plurality of input devices 17 for providing input signals to the system 2 such as one or more control buttons 29, a touch screen 31 and the transceiver 32; (b) a signal generating assembly 16 which produces or generates electronic signals that are received by the transceiver 32; (d) a steering apparatus 79 that receives one or more electronic signals from a power source via the processor 20 or other suitable means; (d) a memory device 21 including machine readable instructions and one or more computer programs (which, for example, may include a plurality of algorithms 23) which are used by the processor 20 to instruct the steering apparatus 79 to change direction as well as to process the signal data produced by the signal generating assembly 16 and transmitted by the transceiver 32; and (e) a plurality of output devices 19 such as the display device 22 and the printer 28 which indicate the catheter information to the health care provider. For instance, the display device 22 and/or the printer 28 can display graphics 37 that help the user in guiding the catheter tip 60 to a desired location within the human body. The display device 22 may be any suitable display mechanism including, but not limited to, a liquid crystal display (LCD), light-emitting diode (LED) display, cathode-ray tube display (CRT) or plasma screen.

In one particular embodiment, the memory device 21 can store instructions which, when executed by the processor 20, cause the processor 20 to (i) interpret catheter 50 location and/or position information as determined and communicated by the signal generating assembly 16 and the non-invasive transceiver 32, and (ii) cause the processor 20 to then instruct the steering apparatus 79 to change direction based on such location and/or position information so that the catheter 50 is steered or guided via the system 2 to a desired anatomical region.

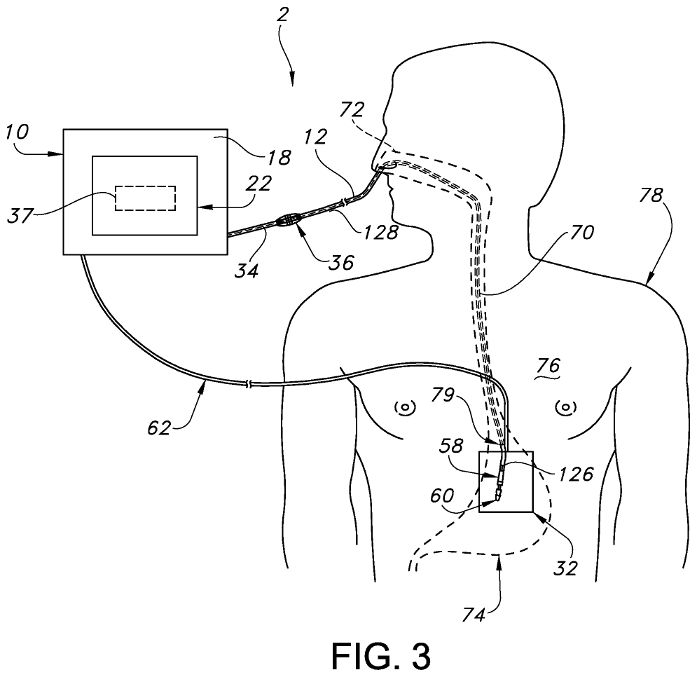

Health care providers can use the system 2 in a variety of catheter applications. In one example illustrated in FIG. 3, the system 2 is used in an enteral application. Here, a portion 70 of the electronic catheter unit 12 is placed through the patient's nose or mouth 72. The distal end or tip 60 of the unit 12 is positioned in the stomach 74, where the distal end or tip 60 of the unit 12 is steered or guided into position via the steering apparatus 79. The health care provider places the transceiver 32 over the chest area 76 of a body 78. The display device 22 and/or the printer 28 can indicate information related to the location of the portion 70 of the electronic catheter unit 12 within the body 78, as well as information related to the shape of the pathway taken by the catheter unit 12. It should be appreciated that the system 2 need not indicate the exact location or path of the catheter unit 12 to provide assistance to the health care provider.

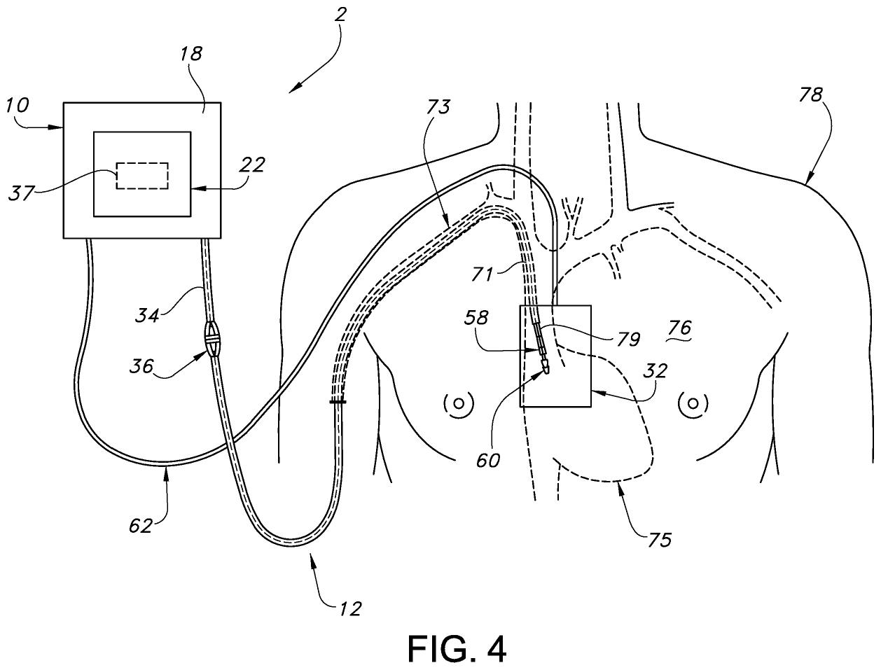

In another example illustrated in FIG. 4, a portion 71 of the electronic catheter unit 12 is introduced into the patient's body 78 through a vein or artery 73 leading to the heart 75. Similar to the enteral example, the system 2 assists the health care provider in guiding the portion 71 of the unit 12 in the patient's vein or artery 73 to a desired cavity in the heart 75 in preparation for parenteral feeding.

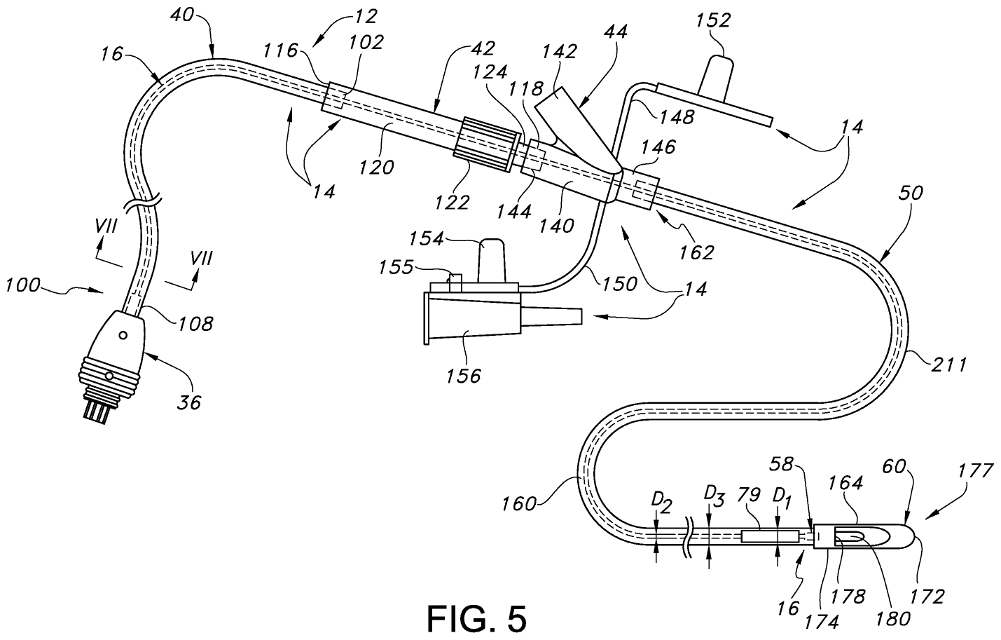

Referring to FIG. 5, in one embodiment, the electronic catheter unit 12 includes a tubing assembly 14 that includes a catheter 50 housing the steering apparatus 79 and signal generating assembly 16. The various components of the electronic catheter unit 12 are discussed in more detail below.

As best illustrated in FIGS. 1 and 5-7, in one embodiment, the tubing assembly 14 includes: (a) a tube or an electrical tubular insulator 40; (b) a mid-connector or union device 42 which receives the tubular insulator 40; (c) a connector 44 attachable to the union device 42 or attachable directly to the tube or electrical insulator 40 without the requirement of the union device 42, where the connector 44 can be a multi-port connector, a y-port connector, or a single port connector; (d) a catheter 50, such as a feeding tube, connected to the connector 44; (e) a catheter end, bolus or tip 60 attached to the distal end of the catheter 50; and (f) a steering apparatus 79 containing an electroactive polymer layer 82 that can adjust the direction in which the catheter 50 travels upon activation.

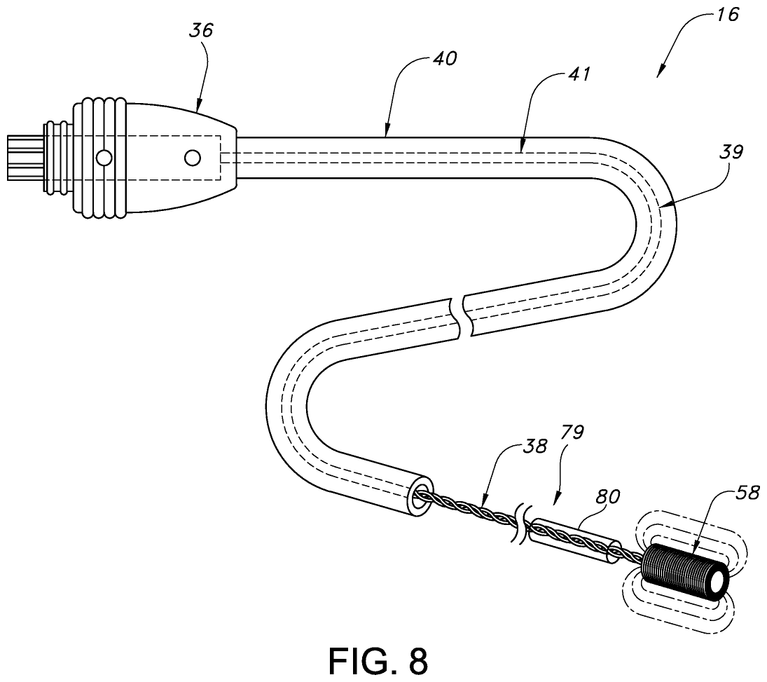

In one embodiment, the tubular insulator 40 includes a tube having: (a) a proximal end 100 attachable to an attachment member or neck 108 of the electronic catheter unit 12; (b) a distal end 102 receivable by the union device 42; (c) an internal diameter which is substantially equal to or greater than an external diameter of a wire assembly 38 (see FIGS. 1 and 8) described below so as to slide over the wire assembly 38; and (d) an external diameter. In another embodiment, the tubular insulator 40 may fit relatively tightly over the wire assembly 38 so as to be secured to the wire assembly 38.

In one embodiment, the union device 42 includes: (a) a proximal end 116; (b) a distal end 118; (c) a position adjuster, extender or elongated neck 120 positioned between the proximal end 116 and the distal end 118; (d) a grasp or gripping member 122 positioned adjacent to the distal end 118 so as to assist users in grasping and manipulating the union device 42; and (e) an insert 124 positioned adjacent to the gripping member 122 which is received by the y-port connector 44. When assembled, the proximal end 116 of the union device 42 is coupled to the distal end 102 of the tubular insulator 40.

In one embodiment, the multi-port or y-port connector 44 includes: (a) a body 140; (b) a liquid delivery branch, medicine delivery branch or medicine branch 142 attached to the body 140 for distributing drugs, medicine or other medicinal liquids to the patient; (c) a nutrient delivery branch or feeding branch 144 attached to the body 140 and sized to receive the insert 124 of the union device 42; (d) a catheter or feeding tube connection branch 146 attached to the catheter 50; (e) a flexible or movable arm 148 attached to the body 140; and (f) a flexible or moveable arm 150 attached to the body 140. In an alternative embodiment, y-port connector 44 includes additional branches for administering various nutrients or medicines to the body 78. In another alternative embodiment, the y-port connector 44 includes only a feeding branch 144 and a connection branch 146. The arm 148 has a stopper 152, and the arm 150 has a stopper 154. The stoppers 152 and 154 are sized to prevent fluid from passing through the branches 142 and 144 after such branches 142 and 144 are plugged with stoppers 152 and 154, respectively. In addition, the arm 150 can include a fastener 155 which secures a tube-size adapter 156 to the arm 150. The tube-size adapter 156 enables fluid delivery tubes (not shown) having various diameters to connect to the feeding branch 144 of the y-port connector 44.

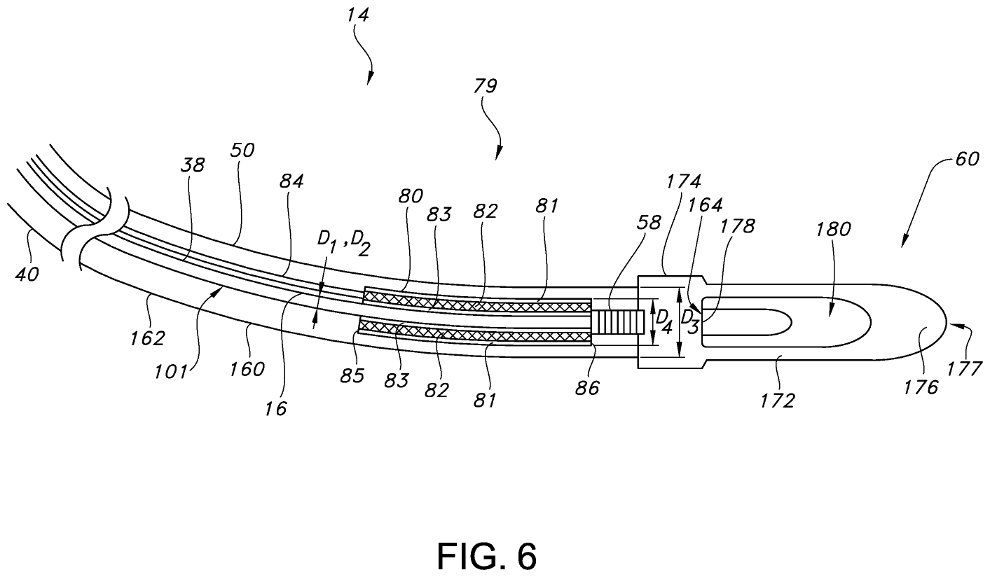

As illustrated in FIGS. 5-6, in one embodiment, the catheter 50 includes a feeding tube with a body 160 having: (a) a proximal end 162 attached to the catheter connection branch 146 of the y-port connector 44; and (b) a distal end 164. The proximal end 162 is insertable into the catheter connection branch 146 of the y-port connector 44 so as to bring the catheter 50 into fluid communication with the y-port connector 44. In one embodiment, the end member, bolus or tip 60 of the catheter 50 is attached to the distal end 164 of the catheter 50. The tip 60 includes a body 172 having a collar 174 and an end member 176. The body 172 defines a passage 178 and an opening 180. The opening 180 is positioned between the collar 174 and the end member 176. A portion 177 of the end member 176 can have a rounded shape. The shape of the passage 178 and opening 180 of the tip 60 is configured to facilitate the flow of fluid from the catheter 50 into the patient's body while decreasing the likelihood that the opening 180 will become clogged.

The tubular connector 40, union device 42, y-port connector 44, catheter 50, and tip 60 can be made from any suitable polymer or plastic material including, but not limited to polyamide, polyethylene, polypropylene, polyurethane, silicone, polyacrylonitrile, poly-L-lysine, poly-D-lysine, polyethylene glycol, polyvinyl alcohol, polyvinyl acetate, polymethyl methacrylate, or a combination thereof.

Further, as illustrated in FIGS. 5-7, the tubing assembly 14 also includes a steering apparatus 79 that can include a sheath 80 or tubing containing one or more polymeric layers 81, 82, and 83 as well as an electrical connection 84. Although a sheath 80 is described in detail below, it is to be understood that the steering apparatus 79 can also be in the form of a coating, wrap, or any other substrate configured for activation of one or more electroactive polymers via an electrical signal delivered via an electrical connection 84 that can be wired (shown) or wireless connection (not shown). For example, the steering apparatus 79 can include a sheath 80 defining a proximal end 85 and a distal end 86 to define an opening or lumen 98 therebetween and can also include an electroactive polymer layer 82. Additionally, in some embodiments, the sheath 80 can include an outer layer 81 that surrounds an outer surface of the electroactive polymer layer 82, where the outer layer 81 helps to insulate the body 78 from any effects of the electroactive polymer layer 82 when it is activated via the electrical connection 84 at the proximal end 85 of the sheath 80. In addition, in some embodiments, the sheath 80 can include an inner layer 83 that surrounds an inner surface of the electroactive polymer layer 82, where the inner layer 83 can insulate the wire assembly 38 and signal generator 58 (discussed in more detail below) from the electroactive polymer layer 82 when the electroactive polymer layer 82 is activated to reduce any interference between the electrical and/or electromagnetic signals associated with each of the individual components. Although any suitable insulating material can be used, in some embodiments, the outer layer 81 and/or the inner layer 83 can include polyamide, polyethylene, polypropylene, poly-L-lysine, poly-D-lysine, polyethylene glycol, polyvinyl alcohol, polyvinyl acetate, polymethyl methacrylate, or a combination thereof. Further, as shown, the proximal end 103 of the electrical connection 84 portion of the steering apparatus 79 is connected to a controller coupler or an electrical connection 36 that is connected to the electrical extension 34, while the distal end 105 of the electrical connection 84 is connected to the proximal end 85 of the sheath 80 of the steering apparatus 79.

Regardless of the particular arrangement of the steering apparatus 79, whether it be in the form of a sheath 80 as described above or as a coating, wrap, etc., the steering apparatus 79 contains an electroactive polymer layer 82 or other electroactive polymer component that can adjust the direction in which the catheter 50 travels upon activation of the electroactive polymer or polymers contained therein. Although any suitable electroactive polymer or combination thereof can be used in the steering apparatus 79 contemplated by the present invention, in some embodiments, the electroactive polymer can be an ionic electroactive polymer such as a conductive polymer, an ionomeric polymer-metal composite (IPMC), or carbon nanotubes (CNT). For example, the conductive polymer can include polypyrrole, poly(3,4-ethylenedioxythiophene), polythiophene, polyaniline, poly-p-phenylene-sulphide, polyacetylene, polyisoprene, polybutadiene, or a combination thereof. Meanwhile, the IPMC can include perfluorosulphonate, perfluorocarboxylate, or a combination thereof. In other embodiments, the electroactive polymer can be an electric electroactive polymer. For instance, in one embodiment, the electric electroactive polymer can be a piezoelectric polymer, such as polyvinylidene fluoride (PVDF) or a copolymer thereof. In another embodiment, the electric electroactive copolymer can be an electro-statically stricted polymer (ESSP), such as polyurethane, silicone, fluorosilicone, fluorodastomer, polybutadiene, isoprene natural rubber latex, polyacrylonitrile, or a combination thereof. In other embodiments, the electric electroactive polymer can be an electrorestrictive graft elastomer, an electro-viscoelastic elastomer, a liquid crystal elastomer, or a combination thereof.

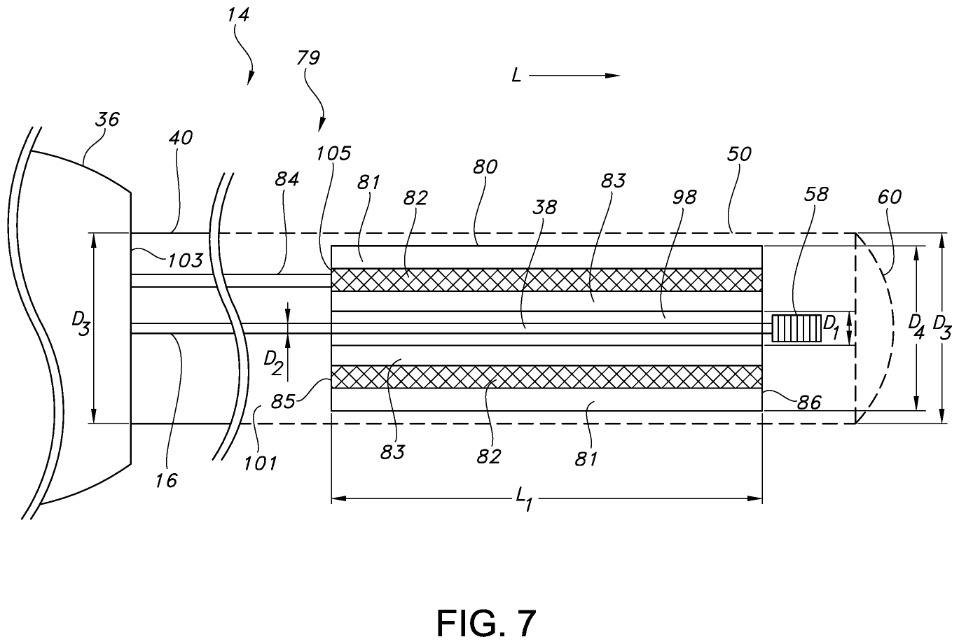

Referring specifically to FIGS. 6-7, when the steering apparatus 79 includes a sheath 80, the sheath can span a length L₁ in the longitudinal direction L in which the tubing assembly 14 and catheter 50 extend ranging from about 0.5 inches (about 12.5 millimeters) to about 10 inches (about 250 millimeters), such as from about 1 inch (about 25 millimeters) to about 8 inches (about 200 millimeters), such as from about 2 inches (about 50 millimeters) to about 6 inches (about 150 millimeters), or any ranges therebetween. In addition, the sheath 80 can have a diameter D₄ that is less than the diameter D₃ of the catheter 50 so that the sheath 80 can fit within the lumen 101 of the catheter 50. Meanwhile, the lumen 98 of the sheath 80 has a diameter D₁ that is large enough to contain the elongated wire assembly 38 component of a signal generating assembly 16, discussed in more detail below, which has a diameter of D₂.

As shown in FIG. 6, in some embodiments, the diameter D₁ of the lumen 98 of the sheath 80 can be generally the same as the diameter D₂ of the elongated wire assembly 38, which can provide additional stiffness or rigidity to the elongated wire assembly 38, which can, in turn, allow for more precise control in moving the tubing assembly 14 that includes the catheter 50 to the desired location within the body 78. As shown in FIG. 7, in other embodiments, the diameter D₁ of the lumen 98 of the sheath 80 can be larger than the diameter D₂ of the elongated wire assembly 38 to provide a space within the lumen 98, which may help to attenuate the electrical signals associated with the electroactive polymer layer 82 upon activation and reduce interference with the signals traveling through the elongated wire assembly 38, which allow for tracking of the catheter tip 60 via the signal generator 58 component of a signal generating assembly 16, which is discussed in more detail below.

As shown in FIGS. 1, 5, and 8, in one embodiment, the catheter guidance system 2 and tubing assembly 14 can also include an invasive signal generating assembly 16. The signal generating assembly 16 can include: (a) the controller coupler or an electrical connector 36 discussed above and connected to the electrical extension 34; (b) an elongated wire assembly 38 connected to the controller coupler or electrical connector 36; (c) a wire or elongated stiffener 39 attached to the controller coupler or electrical connector 36 and serving as a support for the wire assembly 38; (d) a signal generator or magnetic field generator 58 coupled to the distal end of the wire assembly 38; and (e) a suitable fastener attaching the distal end of the elongated stiffener 39 to the magnetic field generator 58. Referring to FIG. 8, the tubular insulator 40 described above covers a portion 41 of the wire assembly 38 positioned adjacent to the controller coupler 36, and it is to be understood that the signal generating assembly 16 can be considered a component of the tubing assembly 14 described above.

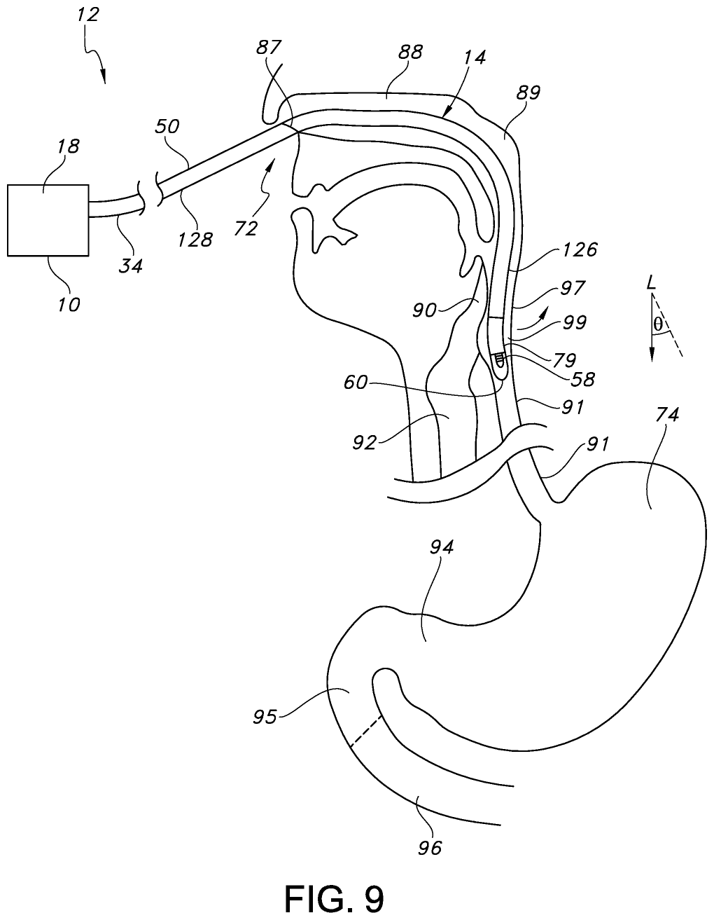

Generally, the controller coupler 36 contains circuitry that enables it to transmit electrical signals (e.g., current and/or voltage) from the apparatus 10 (e.g., the processor or controller 20) through the electrical connection 84 of the steering apparatus 79 to activate the electroactive polymers in the sheath 80 to guide the movement of the catheter 50 within the tubing assembly 14 as it passes through various regions of the body 78. Various methods of steering the tubing assembly 14 to a desired location are discussed in more detail below with respect to FIGS. 9-13. In addition, the same controller coupler 36 can transmit electrical current from the apparatus 10 to the signal generator or magnetic field generator 58 described below to aid in the tracking and/or visualization of the catheter 50 as it is being placed within the body 78.

In one embodiment, the signal generator or magnetic field generator 58 is formed through a plurality of spirals or coils of wire. As the apparatus 10 transmits electrical current through the wires, the current travels in a circular path defined by the coils. This circular motion of current produces an electromagnetic field, B field or electromagnetic radiation. Further, it should be appreciated that the signal generator 58 can include any alternate suitable mechanism or device which generates or produces magnetic energy or a magnetic field. In one embodiment, the magnetic field generator 58 includes a magnet such as a permanent magnet, resistive magnet or superconducting magnet.

In operation, when the apparatus 10 sends electrical current to the coils, the coils transmit a signal or electromagnetic field capable of being detected by the non-invasive transceiver 32. The transceiver 32 then detects the electromagnetic field or signal generated by the signal generator 58 inside the body 78. The processor 20 then causes the display device 22 and/or the printer 28 to produce graphics 37 which assist the health care provider in catheter placement procedure.

Further, although not shown, in an alternative embodiment, it is to be understood that the invasive signal generating assembly 16 including the signal generator 58 can be incorporated directly into tubing assembly 14, for example, by embedding the coils of the signal generator 58 into a wall of the catheter 50.

Methods of using the various assemblies and components described above for accurately placing a catheter 50 in a desired anatomical region in, for example, a gastrointestinal tract of a of a patient are now described in detail with respect to FIGS. 9-13, although it is to be understood that the tubing assembly 14 and other components can be used for placing a catheter in an area outside of the gastrointestinal tract as well.

Generally, a method, such as a computer-implemented method, for steering a catheter 50 during placement of the catheter 50 inside a body 78 of a patient according the present invention, and referring to FIGS. 1, 3, and 9-13, includes inserting a distal end 126 of a tubing assembly 14 into an orifice (e.g., the nose 72) of the body 78. The tubing assembly 14 also has a proximal end 128, which can also be described as the proximal end of the catheter 50, located outside the body 78 and towards the apparatus 10 that can include a controller or processor 20, where the apparatus 10 is connected to the tubing assembly 14 via an electrical extension 34 (e.g., a wire, cable, cord, wireless connection, etc.). Further, the tubing assembly 14 includes the catheter 50, and the steering apparatus 79 is contained within the lumen 101 of the catheter 50 towards the distal end or tip 60 of the catheter, where the tubing assembly 14 containing the catheter 50 and steering apparatus 79 extends in the longitudinal direction L.

Once the distal end 126 of the tubing assembly 14 is inserted into an orifice of the body 78, such as one of the nostrils 87 of the nose 72, various components of the tubing assembly 14 can be connected to the power source 25 such as via the controller coupler 36. For instance, the steering apparatus 79 can be connected to the power source 25 via the electrical extension 34 through the controller coupler 36 and through the electrical connection 84 of the steering apparatus 79. The electrical connection 84 can then be in contact with the electroactive polymer layer 82 of the sheath 80 so that the electroactive polymer layer 82 can be activated as needed via an electrical signal sent by the power source (e.g., current, voltage, etc.) to initiate a change in dimension (e.g., size, shape, diameter, etc.) of the sheath 80, which, in turn, results in the bending or angling of the catheter 50 in a desired direction to guide or steer the catheter 50 to a desired location. Further, although a physical electrical connection between the power source 25 and the steering apparatus 79 via the electrical extension 34 and the electrical connection 84 are described above, it is also to be understood that wireless connections are contemplated by the present invention.

Referring to FIGS. 9-13, the change in dimension of the sheath results in the catheter changing in direction by an angle θ ranging from about 1° to about 180°, such as from about 5° to about 160°, such as from about 10° to about 90°, or any ranges therebetween, relative to the longitudinal direction L in which the catheter 50 extends.

Referring to FIG. 9, in one particular embodiment, the electroactive polymer layer 82 can be activated as the catheter 50 of an enteral feeding tube system in the form of an electronic catheter unit 12 reaches an area near the patient's epiglottis 90 so that the catheter 50 is steered towards the back of the throat 97, enabling the catheter 50 to be properly inserted into the esophagus 91 rather than the trachea 92 after the catheter 50 passes through the nasal cavity 88 and nasopharynx 89. As shown, the catheter 50 can bend in the direction of the arrow as a result of the activation of the electroactive polymer layer 82 of the steering apparatus 79.

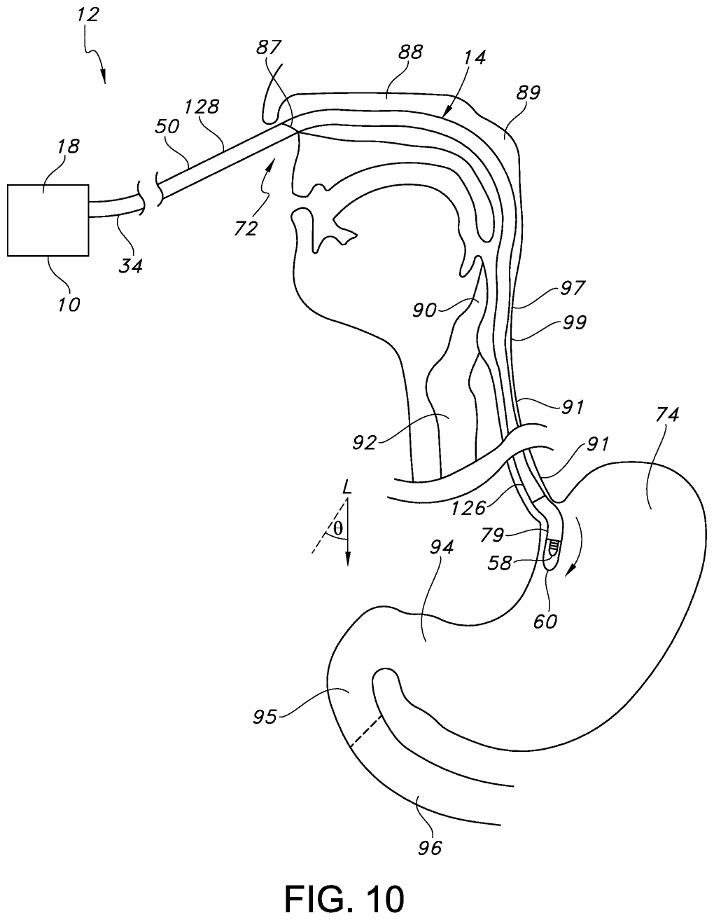

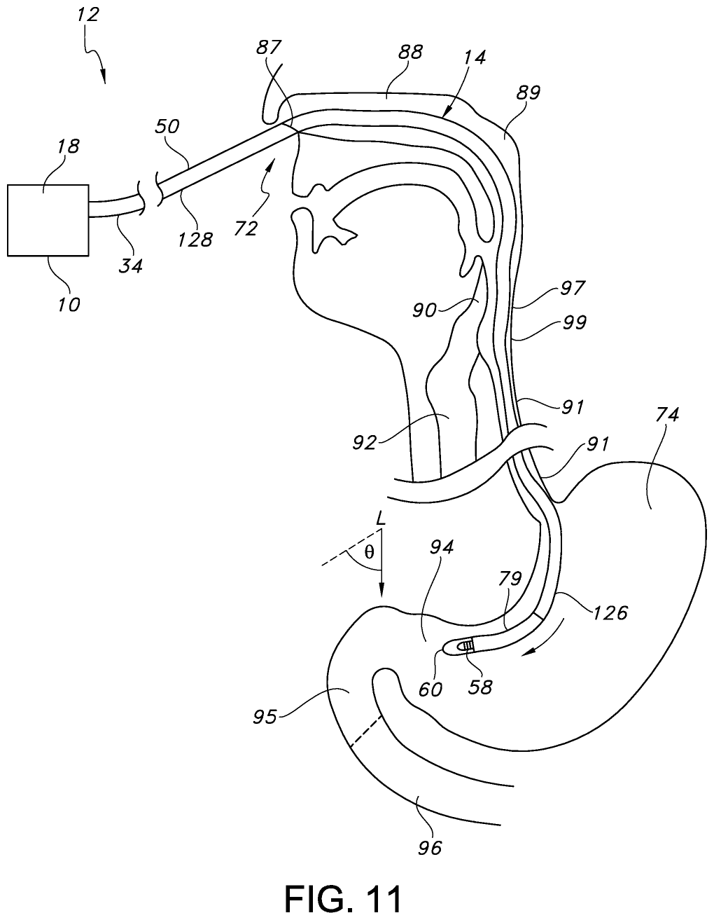

Referring to FIGS. 10 and 11, in another particular embodiment, the electroactive polymer layer 82 can be activated as the catheter 50 of the electronic catheter unit 12 reaches an area near the end of the patient's esophagus 91 so that the catheter 50 is steered towards the pylorus 94 once it enters the stomach 74, enabling the catheter 50 to be properly inserted into the desired location in the gastrointestinal tract. As shown, the catheter 50 can bend in the direction of the arrows in FIGS. 10 and 11 as a result of the activation of the electroactive polymer layer 82 of the steering apparatus 79.

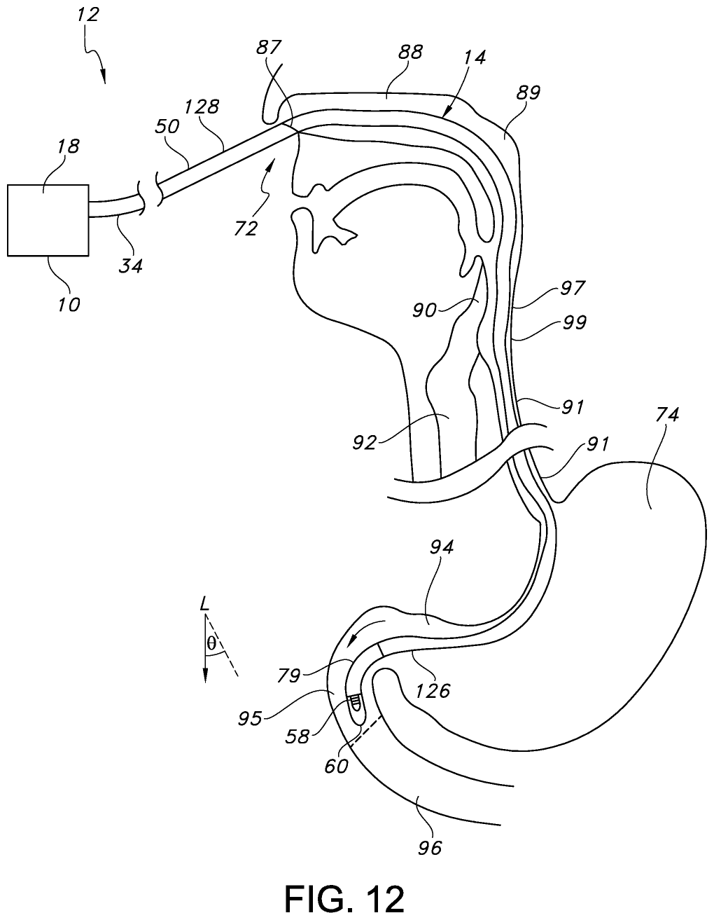

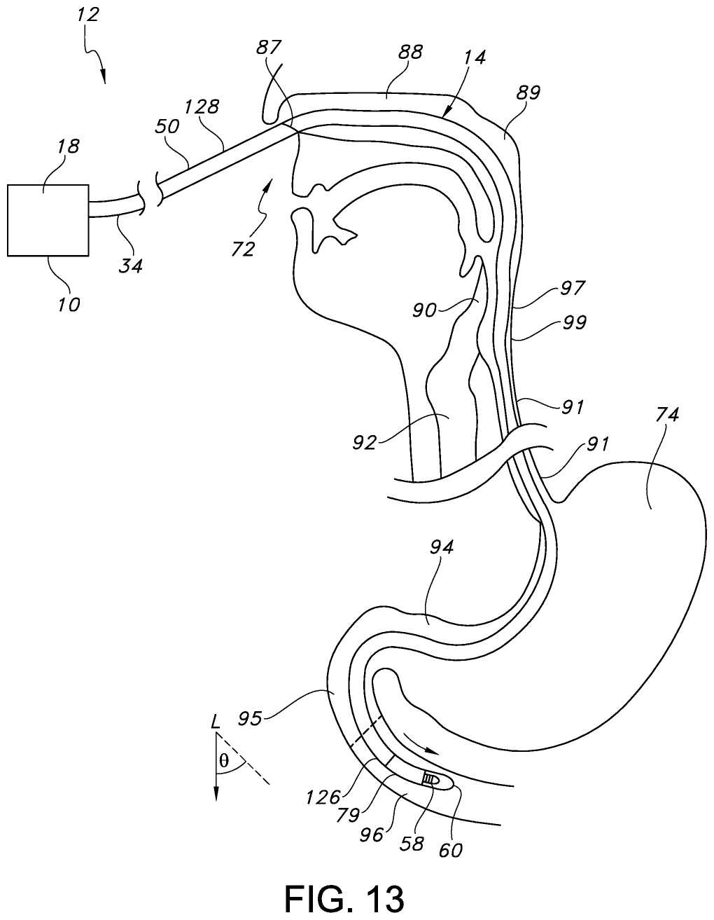

Meanwhile, referring to FIGS. 12 and 13, in other embodiments, the electroactive polymer layer 82 can be activated as the catheter 50 of the electronic catheter unit 12 reaches an area near pylorus 94 after it enters the stomach 74 so that the catheter 50 is properly inserted into the desired location (e.g., the duodenum 95 in FIG. 12 or the jejunum 96 in FIG. 13 in the gastrointestinal tract. As shown, the catheter 50 can bend in the direction of the arrows in FIGS. 12 and 13 to reach the desired locations at the duodenum 95 or the jejunum 96 depending on where the heath care provider has decided to place the catheter 50 for providing nutrition and/or medicine to the patient as a result of the activation of the electroactive polymer layer 82 of the steering apparatus 79.

Further, it should be understood that a method of guiding or steering the placement of the catheter 50 with the steering apparatus 79 as described above with respect to FIGS. 9-13 can be also involve the use of the signal generating assembly 16 including the signal generator 58, where its placement can be controlled to assist in the health care provider in tracking the location of the tubing assembly 14 and steering apparatus 79. In the embodiments best illustrated in FIGS. 1 and 5, the union device 42 assists in maintaining the position of the signal generator 58 at or near the tip 60 of the catheter 50. The use of the union device 42, in one such embodiment, reduces the likelihood that the signal generator 58 might protrude through the tip 60 or stop substantially short of the tip 60. Therefore, the union device 42 functions as a generator placement control device. In one embodiment, this placement and control function of the union device 42 is adjustable to conform to catheters 50 that have different lengths.

In one example, the method of controlling the placement of the signal generator 58 includes first step of determining the length of the catheter 50. Next, prior to placing the catheter 50 into the human body 78 for enteral or parenteral feeding in conjunction with the steering apparatus 79, the health care provider or an assembler can place the signal generator 58 at a desired location within the catheter 50. In one particular embodiment, the sheath 80 of the steering apparatus 79 surrounds the wire assembly 38, while the signal generator 58 is located adjacent the steering apparatus 79 at a distal end 86 of the sheath 80, as shown in FIG. 7. Finally, after proper placement of these components is confirmed, the health care provider or the assembler locks this placement by fastening the tubular insulator 40 to the union device 42 using a suitable adhesive.

Once the position of the steering apparatus 79 and signal generator 58 has been properly set, the health care provider places the transceiver 32 on the patient's chest area 76 and inserts the tubing assembly 14 of the electronic catheter unit 12 including the catheter 50 into the body 78. While doing so, the display device 22 displays graphics 37 that help the user in guiding the catheter tip 60 to a desired location within the human body 78. Once the catheter 50 is placed in the desired location via the aid of the steering apparatus 79 and the signal generating assembly 16 described in detail above, the health care provider can remove the steering apparatus 79 and signal generating assembly 16 including the wire assembly 38 and signal generator 58 while the position of the catheter 50 is maintained. The health care provider can then attach medicine and/or nutritional delivery tubes to the y-port connector 44 for introducing fluids into the body 78 for medical treatment.

It should be appreciated that the tubing assembly, electronic catheter unit and catheter position guidance system of the present invention can be used in a variety of catheter procedures and applications. These procedures may involve the treatment of the gastrointestinal tract, cardiovascular system or other portions of the human body. These procedures may involve treatment of humans by physicians, physician assistants, nurses or other health care providers. In addition, these procedures may involve treatment of other mammals and animals by veterinarians, researchers and others.

The present invention, in one embodiment, includes a tubing assembly and signal generator for an electronic catheter unit of a catheter position guidance system. The tubing assembly and signal generator are used in conjunction with other components of the system to assist the user in performing a catheter placement procedure. The tubing assembly has a position controller which enables the system to be used with catheters of variable lengths. Therefore, the tubing assembly and the position controller, used in conjunction with the catheter position guidance system of the present invention, provide an enhancement in medical treatment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A tubing assembly comprising:
a catheter having a proximal end and a distal end and extending in a longitudinal direction, wherein the proximal end and the distal end define a lumen therebetween; and
a steering apparatus, wherein the steering apparatus comprises an electrical connection having a distal end and a proximal end and a sheath having a proximal end and a distal end and comprising an electroactive polymer layer, wherein the proximal end of the sheath is coupled to the distal end of the electrical connection, wherein the steering apparatus is located within the lumen of the catheter;
wherein activation of the electroactive polymer layer results in a change in dimension of the sheath, wherein the change in dimension initiates a change in a direction in which the catheter travels within a body.

2. The tubing assembly of claim 1, wherein the electroactive polymer layer comprises an ionic electroactive polymer or an electric electroactive polymer.

3. The tubing assembly of claim 1, wherein the sheath further comprises at least one insulating layer.

4. The tubing assembly of claim 3, wherein the sheath comprises a first insulating layer and a second insulting layer, wherein the electroactive polymer layer is disposed therebetween.

5. The tubing assembly of claim 3, wherein the insulating layer comprises polyamide, polyethylene, polypropylene, poly-L-lysine, poly-D-lysine, polyethylene glycol, polyvinyl alcohol, polyvinyl acetate, polymethyl methacrylate, or a combination thereof.

6. The tubing assembly of claim 1, wherein the electrical connection is configured to deliver an electrical signal from a power source to the electroactive polymer layer to initiate the change in dimension.

7. The tubing assembly of claim 1, wherein the change in dimension of the sheath results in the catheter changing in direction by an angle ranging from about 1° to about 180° relative to the longitudinal direction in which the catheter extends.

8. The tubing assembly of claim 1, wherein the proximal end of the electrical connection is coupled to a controller coupler, wherein the controller coupler is configured for connection to a power source.

9. The tubing assembly of claim 1, wherein the electrical connection comprises a wire.

10. The tubing assembly of claim 1, further comprising a signal generating assembly.

11. The tubing assembly of claim 10, wherein the signal generating assembly comprises an elongated wire assembly having a proximal end and a distal end and a signal generator, wherein the signal generator is coupled to the distal end of the elongated wire assembly.

12. The tubing assembly of claim 11, wherein the elongated wire assembly extends through a lumen of the sheath, and wherein the signal generator is positioned adjacent the distal end of the sheath towards the distal end of the catheter.

13. The tubing assembly of claim 11, wherein the signal generator comprises a magnetic field generator.

14. A catheter guidance system comprising:
(a) a controller;
(b) a power source;
(c) a tubing assembly comprising:
a catheter having a proximal end and a distal end and extending in a longitudinal direction, wherein the proximal end and the distal end define a lumen therebetween; and
a steering apparatus, wherein the steering apparatus comprises an electrical connection having a distal end and a proximal end and a sheath having a proximal end and a distal end and comprising an electroactive polymer layer, wherein the proximal end of the sheath is coupled to the distal end of the electrical connection, wherein the steering apparatus is located within the lumen of the catheter; wherein activation of the electroactive polymer layer from the power source via the electrical connection results in a change in dimension of the sheath, wherein the change in dimension initiates a change in direction in which the catheter travels within a body; and
a signal generating assembly; and
(d) a non-invasive movable receiver-transmitter or transceiver in communication with the tubing assembly, wherein the tubing assembly and the non-invasive movable receiver-transmitter or transceiver are electronically coupled to the controller.

15. A method for steering a catheter during placement of the catheter inside a body of a patient, the method comprising:
(a) inserting a distal end of a tubing assembly into an orifice of the body, wherein the tubing assembly also has a proximal end and comprises:
the catheter, wherein the catheter has a proximal end and a distal end and extends in a longitudinal direction, wherein the proximal end and the distal end define a lumen therebetween; and
a steering apparatus, wherein the steering apparatus comprises an electrical connection having a distal end and a proximal end and a sheath having a proximal end and a distal end and comprising an electroactive polymer layer, wherein the proximal end of the sheath is coupled to the distal end of the electrical connection, wherein the steering apparatus is located within the lumen of the catheter;
(b) connecting the tubing assembly to a power source; and
(c) delivering an electrical signal from the power source to the steering apparatus via the electrical connection to activate the electroactive polymer layer, wherein activating the electroactive polymer layer changes a dimension of the sheath, wherein the change in dimension changes a direction in which the catheter travels within the body.

16. The method of claim 15, wherein the orifice is a nose or mouth.

17. The method of claim 15, wherein the change in dimension of the sheath results in the catheter changing in direction by an angle ranging from about 1° to about 180° relative to the longitudinal direction in which the catheter extends.

18. The method of claim 15, wherein the electroactive polymer layer comprises an ionic electroactive polymer or an electric electroactive polymer.

19. The method of claim 15, comprising activating the electroactive polymer layer as the catheter reaches an area near the patient's epiglottis so that the catheter is steered towards the esophagus rather than the trachea.

20. The method of claim 15, comprising activating the electroactive polymer layer as the catheter reaches an area near the patient's pylorus to facilitate duodenal or jejunal placement of the catheter.
